(19) United States
(12) Patent Application Publication
(10) Pub. No.: US 2014/0316268 A1
(43) Pub. Date: Oct. 23, 2014

(54) PERIPHERAL NERVE IDENTIFICATION

(71) Applicants: Ronny Kafiluddi, Albany, NY (US); Ersno Eromo, Ghent, NY (US)

(72) Inventors: Ronny Kafiluddi, Albany, NY (US); Ersno Eromo, Ghent, NY (US)

(21) Appl. No.: 14/174,464

(22) Filed: Feb. 6, 2014

Related U.S. Application Data
(60) Provisional application No. 61/761,258, filed on Feb. 6, 2013.

(51) Int. Cl. A61B 18/12; A61B 5/0492

(52) U.S. Cl. CPC A61B 18/1206; A61B 5/0492; A61B 2018/00434
USPC 600/439; 606/34; 600/546; 600/476

(57) ABSTRACT

A system for accurate identification of a targeted peripheral nerve. The system includes a computing device, a radio frequency generator, and an electromyograph. The system operates to stimulate a stimulating needle, measure signals from muscles innervated by the targeted peripheral nerve, and block electrical activity of the targeted peripheral nerve. In some embodiments the nerve identification is used as part of a nerve ablation procedure or an injection procedure, for example.

### CROSS-REFERENCE TO RELATED APPLICATION

This application claims priority to U.S. Ser. No. 61/761,258, filed on Feb. 6, 2013, and titled PERIPHERAL NERVE IDENTIFICATION, the disclosure of which is hereby incorporated by reference in its entirety.

### TECHNICAL FIELD

This disclosure relates generally to identification of a targeted peripheral nerve in a patient, and more particularly, to accurately identifying the targeted peripheral nerve as part of a nerve ablation or injection procedure.

### BACKGROUND

The medial nerve branch is a peripheral nerve which innervates the facet joints. The medial nerve is a branch of the posterior ramus of the spinal nerve. Apart from innervating the facet joint, the medial nerve branch also innervates the multifidus muscle in the back. The multifidus muscle is located along the laminae of the vertebrae, and its function is to aid in stability of the back.

Chronic back pain may be caused by symptomatic arthritis of the facet joints in the cervical, thoracic or lumbar spine. In order to diagnose whether pain is secondary to the above condition, diagnostic blocks (using local anesthetics) are frequently used. Should the patient's pain subside after blocking the nerve that conducts pain signals from the painful joint or site to the brain, one may conclude that the structure innervated by that particular nerve is the pain generator (in this case the facet joint). Location of the medial branch innervating the facet joint was determined using dissections of cadavers. However, whether the medial nerve branch is actually located at that junction between the above mentioned structures is just an assumption.

Currently, the medial nerve branch of the posterior ramus of the spinal nerve is identified using: (a) fluoroscopic or ultrasonic guidance combined with (b) visual inspection of muscle contractions and (c) subjective experience of similar pain upon stimulation of a targeted nerve. However, each method is limited. For example, fluoroscopically guided needle placement allows the needle to be located roughly in the area of the targeted nerve(s), but does not consider possible anatomical variations of the location of the targeted nerve.

Theoretically, using ultrasonic guidance, peripheral nerves can be visualized and the needle placed in close proximity. However, a lack of resolution makes nerves that are much deeper (e.g., further away from the skin) very difficult to visualize using ultrasound guidance. Additionally, it is difficult to distinguish between a nerve and a ligament in the ultrasound-based imaging. For some patients, such as obese patients, elderly patients with atrophy of the muscles, or the like, muscle contractions upon stimulation of the nerve innervating the muscle frequently are not perceived well. Sensory stimulation also can be inaccurate since it is very subjective and the patient is frequently lightly sedated, thereby impairing his/her ability to experience the sensory stimulation.

Identification of the medial nerve branches of the posterior ramus of the spinal nerves is often performed as part of a neuroablation procedure of the medial nerve branches using a radiofrequency probe. A current approach to circumvent inaccurate placement of the needle is to enlarge the lesion size with, for example, “cold radiofrequency” neurotomies or using multiple filaments. Such a neuroablation procedure has been used for at least thirty years in the treatment of facet joint-related back pain.

### SUMMARY

Aspects of the present disclosure provide a solution for accurately identifying peripheral nerves, e.g., prior to injecting or performing a neuroablative treatment procedure. In addition, aspects of the disclosure provide a solution for assessing the effectiveness of the ablation procedure by repeating an electromyogram (EMG) recording after the treatment. An embodiment of the present disclosure provides a computer system, which can be implemented as a single physical device, capable of: generating stimulation pulses; generating a radio frequency (RF) field; measuring temperature at a tip of an electrode; displaying the measured temperature; maintaining a constant temperature at the tip; measuring and/or recording data corresponding to muscle compound action potentials (CMAPs) and/or motor unit action potentials (MUAPs); correlating the data with a location of a target nerve, a treatment being performed on the target nerve, and/or an effectiveness of the treatment; and/or the like.

Other aspects of the present disclosure provide methods, systems, program products, and methods of using and generating each, which include and/or implement some or all of the actions described herein. The illustrative aspects of the present disclosure are designed to solve one or more of the problems herein described and/or one or more other problems not discussed.

### BRIEF DESCRIPTION OF THE DRAWINGS

These and other features of the disclosure will be more readily understood from the following detailed description of the various aspects of the present disclosure taken in conjunction with the accompanying drawings that depict various aspects of the present disclosure.

FIG. 1 shows an illustrative environment for locating a targeted nerve in a patient according to embodiments.

FIG. 2 shows another illustrative environment for locating a targeted nerve in a patient according to embodiments.

FIG. 3 shows a cross section through a lumbar vertebral level according to an embodiment.

FIG. 4 shows an oblique view of the lumbar spine showing the lumbar vertebrae (skin, tendons, adipose tissue and muscles removed) according to an embodiment.

FIG. 5 shows an illustrative schematic of the electrical components according to an embodiment.

It is noted that the drawings may not be to scale. The drawings are intended to depict only typical aspects, and therefore should not be considered as limiting. In the drawings, like numbering represents like elements between the drawings.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

The inventors recognize a need to more precisely locate a targeted nerve in a patient, e.g., in order to perform a neuroablative treatment procedure.

As indicated above, aspects of the disclosure provide a solution for accurately identifying peripheral nerves, e.g., prior to injecting or performing a neuroablative treatment procedure. In addition, aspects of the disclosure provide a solution for assessing the effectiveness of the ablation procedure by repeating an electromyogram (EMG) recording after the treatment. As used herein, unless otherwise noted, the term “set” means one or more (i.e., at least one) and the phrase “any solution” means any now known or later developed solution.

In the medical field, interventional spine specialists, radiologist, orthopedic surgeons, neurosurgeons, pain management physicians, as well as PM&R specialists routinely provide diagnostic and therapeutic injections with the goal of isolating peripheral nerves. Illustrative aspects of the disclosure are shown and described in conjunction with a solution for the neuroablation of medial nerve branches of the posterior ramus of the spinal nerves of a human patient using a radiofrequency probe. However, it is understood that aspects of the disclosure can be applied to identifying any peripheral nerve of any mammalian patient.

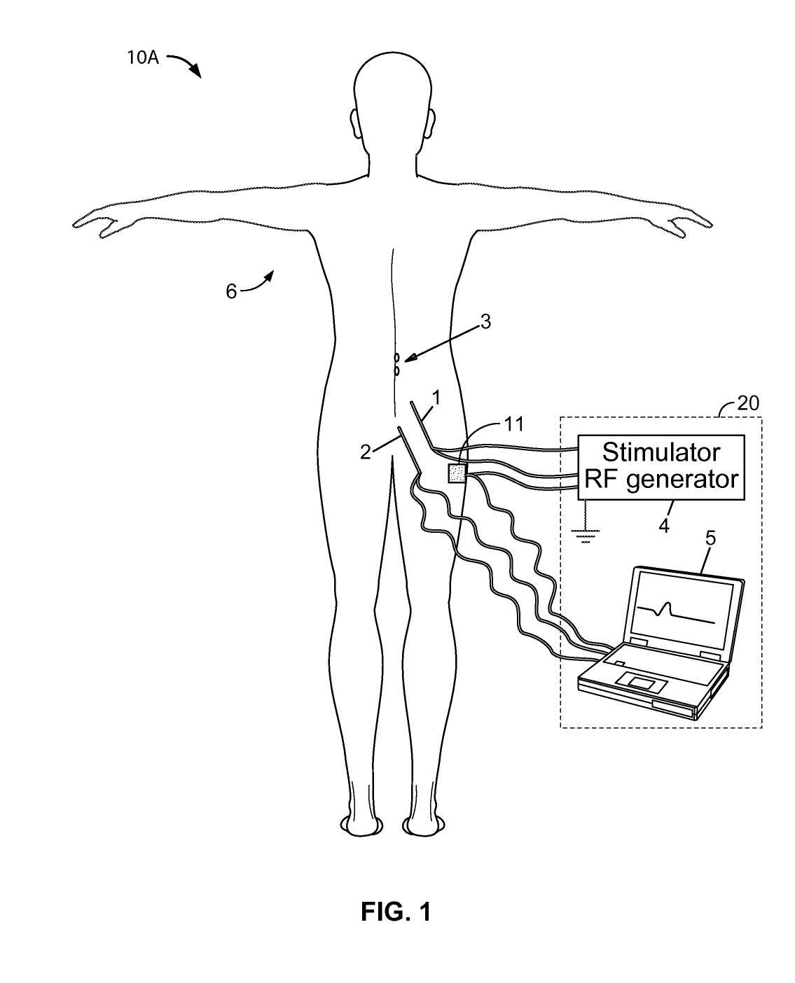

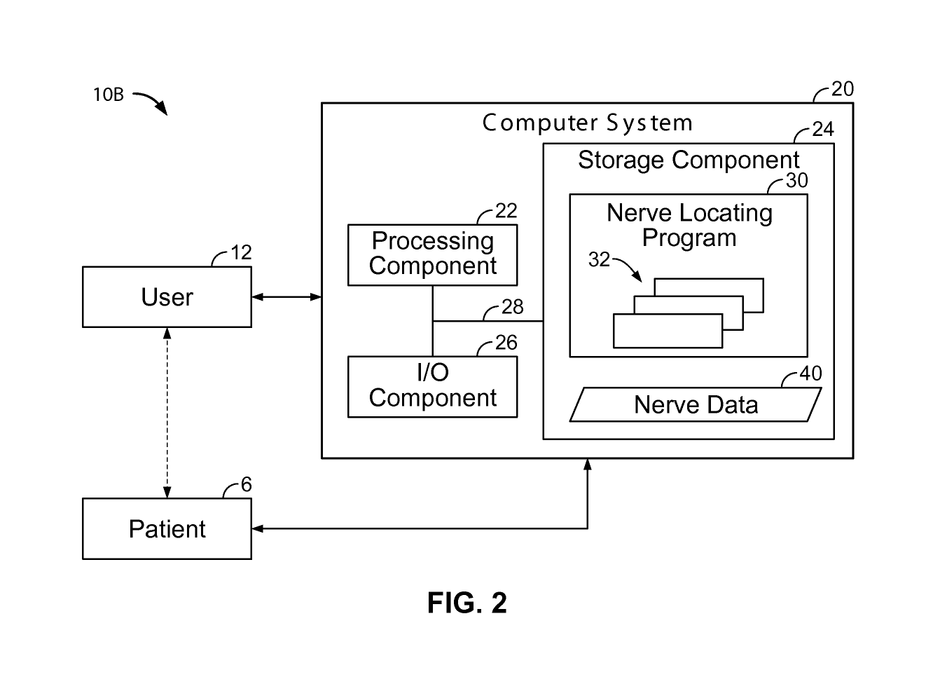

Turning to the drawings, FIGS. 1 and 2 show illustrative environments 10A, 10B for locating a targeted nerve in a patient 6 according to embodiments. To this extent, the environment 10 includes a computer system 20 that can perform a process described herein in order to locate the targeted nerve in the patient 6. In particular, the computer system 20 is shown including a nerve locating program 30, which makes the computer system 20 operable to locate the targeted nerve in the patient 6 by performing a process described herein.

The computer system 20 is shown including a processing component 22 (e.g., one or more processors), a storage component 24 (e.g., a storage hierarchy), an input/output (I/O) component 26 (e.g., one or more I/O interfaces and/or devices), and a communications pathway 28. In general, the processing component 22 executes program code, such as the nerve locating program 30, which is at least partially fixed in storage component 24. While executing program code, the processing component 22 can process data, which can result in reading and/or writing transformed data from/to the storage component 24 and/or the I/O component 26 for further processing. The pathway 28 provides a communications link between each of the components in the computer system 20. The I/O component 26 can comprise one or more human I/O devices, which enable a human user 12 to interact with the computer system 20 and/or one or more communications devices to enable a system user 12 to communicate with the computer system 20 using any type of communications link. To this extent, the nerve locating program 30 can manage a set of interfaces (e.g., graphical user interface(s), application program interface, and/or the like) that enable human and/or system users 12 to interact with the nerve locating program 30. Furthermore, the nerve locating program 30 can manage (e.g., store, retrieve, create, manipulate, organize, present, etc.) the data, such as nerve data 40, using any solution.

In any event, the computer system 20 can comprise one or more general purpose computing articles of manufacture (e.g., computing devices) capable of executing program code, such as the nerve locating program 30, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. To this extent, the nerve locating program 30 can be embodied as any combination of system software and/or application software.

Furthermore, the nerve locating program 30 can be implemented using a set of modules 32. In this case, a module 32 can enable the computer system 20 to perform a set of tasks used by the nerve locating program 30, and can be separately developed and/or implemented apart from other portions of the nerve locating program 30. As used herein, the term “component” means any configuration of hardware, with or without software, which implements the functionality described in conjunction therewith using any solution, while the term “module” means program code that enables a computer system 20 to implement the actions described in conjunction therewith using any solution. When fixed in a storage component 24 of a computer system 20 that includes a processing component 22, a module is a substantial portion of a component that implements the actions. Regardless, it is understood that two or more components, modules, and/or systems may share some/all of their respective hardware and/or software. Furthermore, it is understood that some of the functionality discussed herein may not be implemented or additional functionality may be included as part of the computer system 20.

When the computer system 20 comprises multiple computing devices, each computing device can have only a portion of the nerve locating program 30 fixed thereon (e.g., one or more modules 32). However, it is understood that the computer system 20 and the nerve locating program 30 are only representative of various possible equivalent computer systems that may perform a process described herein. To this extent, in other embodiments, the functionality provided by the computer system 20 and the nerve locating program 30 can be at least partially implemented by one or more computing devices that include any combination of general and/or specific purpose hardware with or without program code. In each embodiment, the hardware and program code, if included, can be created using standard engineering and programming techniques, respectively.

Regardless, when the computer system 20 includes multiple computing devices, the computing devices can communicate over any type of communications link. Furthermore, while performing a process described herein, the computer system 20 can communicate with one or more other computer systems using any type of communications link. In either case, the communications link can comprise any combination of various types of optical fiber, wired, and/or wireless links; comprise any combination of one or more types of networks; and/or utilize any combination of various types of transmission techniques and protocols.

As discussed herein, the nerve locating program 30 enables the computer system 20 to locate a targeted nerve in the patient 6. To this extent, further aspects of the disclosure are described in conjunction with the treatment of a peripheral nerve, e.g., to address a painful facet joint that is innervated by a medial nerve branch of a posterior ramus of the spinal nerves. Aspects of the disclosure can be used for ablation of the cervical, thoracic, and lumbar medial nerve branches of the posterior ramus of the spinal nerves, occipital nerves, sacral nerves and other peripheral nerves. However, it is understood that aspects of the disclosure can be utilized in conjunction with any type of treatment.

As shown in FIG. 1, the computer system 20 can include a radio frequency generator (4) and an electromyograph, e.g., presented by a computing device (5). These components can be conventional components, or a single physical device including a combination of the two components can be used.

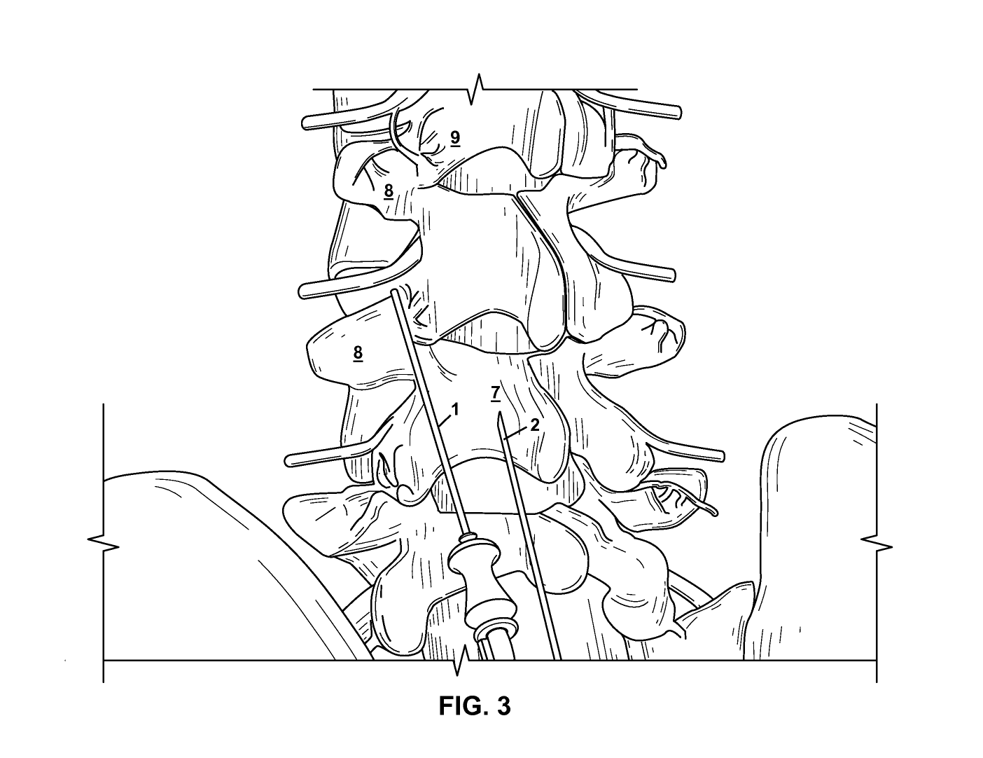

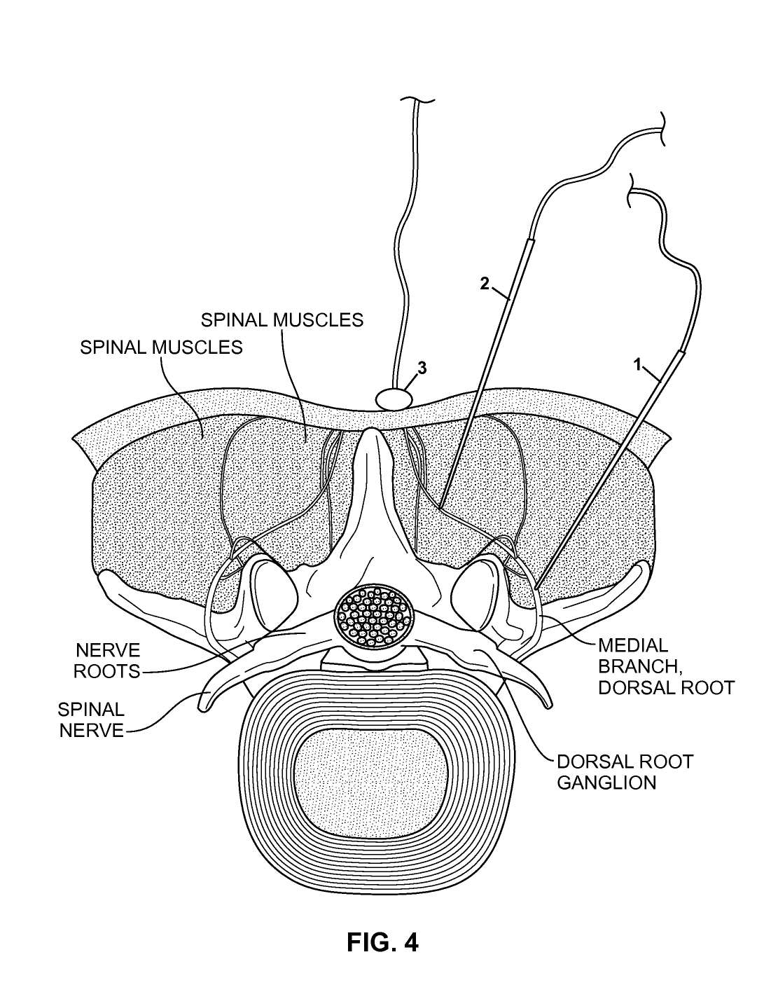

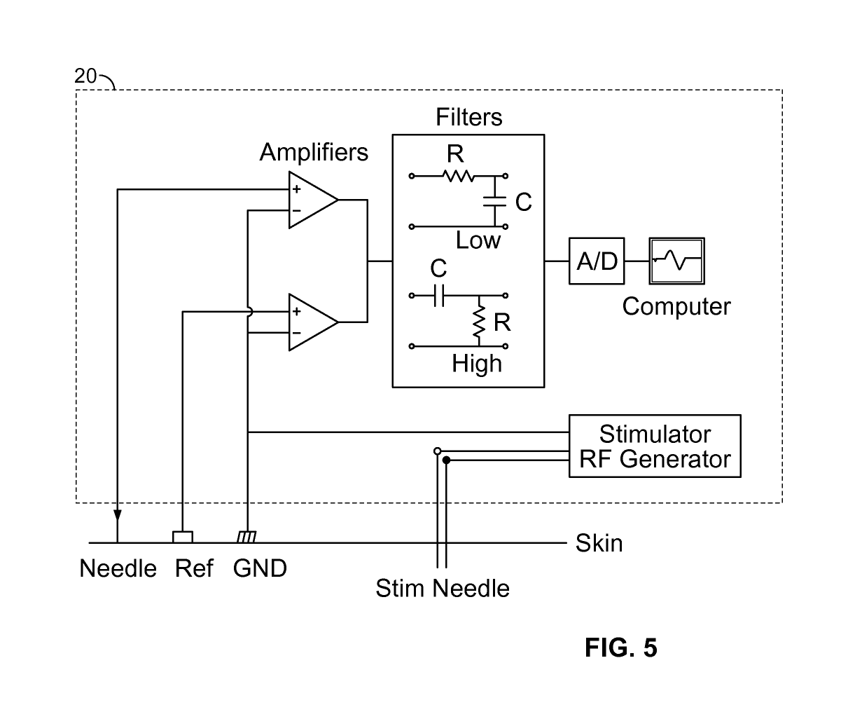

Regardless, in an embodiment, two needles can be used. In an illustrative process performed using the computer system 20, once the patient 6 is placed in the prone position and prepped with an antiseptic solution, the facet joints and spinous processes are localized using a fluoroscope (which can be included as part of the computer system 20). Needle entry sites on the patient 6 can be anesthetized using a small amount of local anesthetic solution. As shown more clearly in FIGS. 3 and 4, a recording needle (2) can be advanced and placed in the muscle belly of the multifidus muscle located paramedial to the spinous processes. The connecting wires from this recording electrode (2) can be attached to the computer system 20, which can include, as shown in FIG. 5, for example, a conventional pre-amplifier fed into an analog/digital (A/D) converter and into a computer (e.g., a laptop) containing the necessary software to analyze the receiving signals.

Returning to FIGS. 3 and 4, a stimulating needle (1), which can be used to stimulate, inject and/or pass radiofrequency waves in order to electrically ablate the nerve, can be directed towards the nerve the user 12 is interested in identifying. The position of this needle (1) relative to the nerve is assessed using signals from the muscle (multifidus) the (medial) nerve innervates using a second needle (2) either placed directly into the muscle or skin electrodes (2) able to record signals from the muscle. The first needle (1) can be guided using fluoroscopic guidance in a patient 6, who is lying in the prone position. The needle (1) can be placed at a junction between the transverse process (8) of the vertebra and the superior articular process (9) of the lamina of the vertebra (7) with the presumed painful facet joint.

Once periostium is reached, the stimulating needle (1) can be connected to a nerve stimulator of the computer system 20 (e.g., as shown in FIG. 5). The stimulating part of the needle can include a conducting electrode tip (e.g., approximately 5-10 mm long) which is located at the distal end of an insulated needle. Square wave electrical pulses (1-10 ms in duration) and with a frequency of two Hz can be generated by the nerve stimulator (4) shown in FIG. 1.

Returning to FIGS. 1 and 2, the electromyograph (5) can be used to localize the peripheral nerve and the radio frequency generator (4) can be used to perform a neurotomy (nerve ablation). In an embodiment, the nerve to be ablated (e.g. the medial branch of the posterior ramus of the spinal nerve) innervates a muscle (e.g. the multifidus muscle in the back or neck). Upon stimulation of the medial nerve, the multifidus muscle will contract because action potentials generated by stimulation of the parent nerve (medial nerve branch) will travel distally and activate the muscle to contract.

The stimulating part of the needle (1) can include a conducting electrode tip (e.g., approximately 5-10 mm long), which is located at a distal end of an insulated needle (1). The radio frequency generator (4) can generate square wave electrical pulses (e.g., approximately 1-10 ms in duration) with a frequency of approximately two hertz (Hz). Once the stimulating needle (1) is placed in close proximity to the medial nerve branch, e.g., using fluoroscopic guidance, the radio frequency generator (4) can increase the stimulation voltage until a threshold is reached, which elicits an action potential. Muscle activity (from the multifidus muscle) can be observed on an electromyography (5) generated by the computer system 20.

As described herein, either a surface or intramuscular recording electrode (3) can be used to record the muscle activity after stimulation of the parent nerve. The computer system 20 can measure muscle compound action potentials (CMAPs) using a surface recording electrode (3), and/or the computer system 20 can measure motor unit action potentials (MUAPs) using a recording needle (3) directly placed into the muscle belly (e.g., a concentric or monopolar recording electrode). The closer, anatomically, the stimulating needle (1) is to the medial nerve, the more muscle fibers will be recruited and the larger the measured CMAPs and/or MUAPs will be. Maximal signal strength can correspond to the closest anatomical placement with regard to the nerve, thereby providing a more accurate identification of the location of the medial nerve.

The stimulating needle (1) can be manipulated such as to get closer to the nerve by observing the size of the elicited MUAPs or CMAPs from the innervated muscle (e.g., depending on whether an intramuscular recording needle of skin electrodes are used) using the computer system 20. Once the nerve has been accurately localized using fluoroscopy (and/or using ultrasound guidance) and/or physiologically (e.g., electrically as described herein), one can either block the nerve using a local anesthetic as diagnostic block or produce a radiofrequency lesion (e.g., in order to block the nerve for a much longer period of time, usually 8-14 months).

As a result, contrary to prior approaches, aspects of the disclosure enable the identification of a targeted nerve using visual guidance, e.g., by using fluoroscopy as well as physiologic (electrical) means to localize the nerve using electromyographic evidence. With the prior approaches, an individual is never completely sure whether the actual nerve has been physiologically blocked. In contrast, aspects of the current disclosure enable a user to have a high degree of certainty. In addition, once the targeted nerve has been identified using a combination of fluoroscopic (and/or ultrasound) and electromyographic information, the computer system 20 can be used to perform an electromagnetic radiofrequency ablation (e.g., usually at approximately eighty degrees Centigrade for approximately ninety seconds) in order to obtain longer lasting pain relieve from the painful facet joints.

In summary, in an embodiment of the current disclosure, a nerve stimulator attached to a stimulating needle and an electromyograph attached to recording electrodes are used for more precise localization of a targeted nerve, e.g., to perform a diagnostic (temporarily) nerve block, a radiofrequency ablation (longer lasting block), and/or the like. Some advantages, which can be obtained by embodiments of the disclosure, include: (i) accurate localization of peripheral nerves; (ii) demonstrating post ablation/neurotomy electrical changes in the recorded CMAPs or MUAPs; (iii) avoiding nerve damage to other sensory or motor nerves; (iv) savings due to incidences of false positive diagnostic blocks (approximately 30% using the prior approaches) being drastically reduced; (v) post procedure assessment of the effectiveness of the performed block or ablation; and/or the like.

FIG. 5 shows a more particular illustrative embodiment of the disclosure. As shown, the computer system 20 can comprise a combination of an electromyograph, which assists in localizing the nerve to be blocked or ablated, and a radiofrequency generator. Once the nerve is localized, the nerve can be temporarily blocked by injecting a local anesthetic solution or ablated using the radiofrequency generator. The electromyography can register signals obtained from muscle activity upon stimulation of the nerve with a nerve stimulator. Muscle activity is recorded using surface electrodes (CMAPs) and/or intramuscularly placed electrodes (MUAPs). The recording electrodes can be connected to a pre-amplifier, high frequency and low frequency band filters, an A/D converter and a common mode rejection amplification system. The filtered digital signals can be fed into a computing device, which displays the obtained signals and performs basic operations on these CMAP or MUAP signals (Fast Fourier Transformation and differentiation).

The target nerve can be stimulated using a nerve stimulating needle. Through the needle, a probe is connected to a nerve stimulator. The nerve stimulator can be a conventional stimulator (e.g., able to inject current with a certain pulse width, frequency and amplitude) or alternatively, the nerve stimulator can be encased in a conventional radiofrequency generator. The latter generates the heat necessary to ablate the nerve using very high frequency pulses (500 MHz) at the tip of the stimulating needle. In addition, a thermocouple can be placed at the tip of the stimulating needle, which can provide data to enable the heat produced by the radiofrequency generator to be monitored by the computer system 20. The temperature at the tip of the needle can be controlled by the computer system 20 using differential feedback amplification. A common grounding electrode (e.g., a grounding pad (11) as shown in FIG. 1) can be connected to the patient 6 and to the nerve stimulator, electromyograph, and the patient.

As described herein, the stimulating needle can be advanced through the anesthetized skin and directed using fluoroscopic guidance towards the nerve to be blocked or ablated. The needle can be subsequently connected to the stimulator or radiofrequency generator containing a stimulator module. In addition, either surface electrodes or a conventional concentric or monopolar needle can be advanced and placed into the muscle belly innervated by the nerve. In the case of the medial nerve of the posterior ramus of the spinal nerve, the muscle would be the multifidus, which is paraspinally adjacent to the spinous processes. The intramuscular needle can be placed slightly proximal to the lamina of the vertebra (needle placement visualized under fluoroscopy). The recording electrodes can be connected to the electromyography and impedances measured by the computer system 20 in order to verify the absence of open circuits.

Stimulation can be initiated by the computer system 20 and CMAPs and/or MUAPs observed on a computer display. The computer system 20 can perform and/or obtain data analysis using the power spectrum derived from the Fast Fourier Transformation of the signals in order to verify that the signals to be analyzed are from a two Hz stimulation frequency and not from noise. The computer system 20 also can adjust the filter settings in order to fine tune the signals. The position of the stimulating needle subsequently can be slightly changed anatomically and the amplitude of the action potentials can be observed using the computer system 20. In addition, the computer system 20 can calculate maxima of waves, e.g., by taking derivatives, and display the maxima as a function of time while the stimulating electrode is manipulated. A location of the stimulating electrode at which the amplitude is maximal is the anatomically appropriate location of the nerve.

At this point, one may choose to block the nerve using a local anesthetic solution or to use the radiofrequency generator of the computer system 20 to ablate the nerve. The ablation is usually done by setting a temperature of 80 degrees Celsius and ablating the nerve for 60 to 90 seconds. The computer system 20 can repeat the stimulation after the nerve block or nerve ablation in order to assess whether the CMAPs or MUAPs are indeed diminished. This will prove the effectiveness of the block or lesion and verify that the nerve was indeed physiologically and anatomically correctly identified.

While primarily shown and described herein as a method and system for locating a target nerve, it is understood that aspects of the disclosure further provide various alternative embodiments. For example, in one embodiment, the disclosure provides a computer program fixed in at least one computer-readable medium, which when executed, enables a computer system to locate the target nerve. To this extent, the computer-readable medium includes program code, such as the nerve locating program 30 (FIG. 2), which enables a computer system to implement some or all of a process described herein. It is understood that the term “computer-readable medium” comprises one or more of any type of tangible medium of expression, now known or later developed, from which a copy of the program code can be perceived, reproduced, or otherwise communicated by a computing device. For example, the computer-readable medium can comprise: one or more portable storage articles of manufacture; one or more memory/storage components of a computing device; paper; and/or the like.

In another embodiment, the present disclosure provides a method of providing a copy of program code, such as the nerve locating program 30 (FIG. 2), which enables a computer system to implement some or all of a process described herein. In this case, a computer system can process a copy of the program code to generate and transmit, for reception at a second, distinct location, a set of data signals that has one or more of its characteristics set and/or changed in such a manner as to encode a copy of the program code in the set of data signals. Similarly, an embodiment of the disclosure provides a method of acquiring a copy of the program code, which includes a computer system receiving the set of data signals described herein, and translating the set of data signals into a copy of the computer program fixed in at least one computer-readable medium. In either case, the set of data signals can be transmitted/received using any type of communications link.

In still another embodiment, the present disclosure provides a method of generating a system for locating a target nerve. In this case, the generating can include configuring a computer system, such as the computer system 20 (FIGS. 1 and 2), to implement the method of locating a target nerve. The configuring can include obtaining (e.g., creating, maintaining, purchasing, modifying, using, making available, etc.) one or more hardware components, with or without one or more software modules, and setting up the components and/or modules to implement a process described herein. To this extent, the configuring can include deploying one or more components to the computer system, which can comprise one or more of: (1) installing program code on a computing device; (2) adding one or more computing and/or I/O devices to the computer system; (3) incorporating and/or modifying the computer system to enable it to perform a process described herein; and/or the like.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

1-4. (canceled)

5. A system for identifying a targeted peripheral nerve in a patient, the system comprising:
a stimulating needle; and
a computer system including at least one device that operates to:
stimulate the stimulating needle;
measure signals from muscles innervated by the targeted peripheral nerve; and
block electrical activity of the targeted peripheral nerve.

6. The system of claim 5, further comprising a recording needle that is configured to record the signals from the muscles.

7. The system of claim 5, wherein the computer system includes an electromyograph.

8. The system of claim 7, wherein the electromyograph is attached to recording electrodes.

9. The system of claim 5, wherein the computer system includes a radio frequency generator.

10. A method for identifying a targeted peripheral nerve in a patient, the method comprising:
generating a first signal at a distal end of a first needle at a desired position in a patient, wherein the first signal stimulates a nerve in proximity to the distal end of the first needle;
causing the nerve in proximity to the distal end of the first needle to innervate a muscle; and
receiving a second signal from the muscle, the second signal being associated with muscle activity.

11. The method of claim 10, further comprising repositioning the first needle to a second desired position, the second desired position being associated with a more accurate localization of the nerve.

12. The method of claim 10, further comprising blocking the nerve with the distal end of the first needle.

13. The method of claim 12, wherein blocking comprises passing radio frequency waves through the nerve.

14. The method of claim 10, wherein guiding comprises using a fluoroscope to visually position the first needle.

15. The method of claim 10, wherein guiding comprises using ultrasound to visually position the first needle.
